(19) United States
(12) Patent Application Publication
(10) Pub. No.: US 2016/0051162 A1
(43) Pub. Date: Feb. 25, 2016

(54) METHOD FOR LOCATING A BRAIN ACTIVITY ASSOCIATED WITH A TASK

(71) Applicant: Commissariat a L'Energie Atomique et aux Energies Alternatives, Paris (FR)

(72) Inventors: Pierre DURAND, La Tronche (FR); Etienne LABYT, Saint Martin Le Vinoux (FR)

(73) Assignee: Commissariat a L'Energie Atomique et aux Energies Alternatives, Paris (FR)

(21) Appl. No.: 14/809,719

(22) Filed: Jul. 27, 2015

(30) Foreign Application Priority Data
Jul. 29, 2014 (FR) 14 57325

(51) Int. Cl. A61B 5/0484; A61B 5/00; A61B 5/04

(52) U.S. Cl. CPC A61B 5/0484; A61B 5/04012; A61B 5/7203; A61B 5/04009

(57) ABSTRACT

The invention relates to a method for estimating the electrical activity of a tissue using a plurality of sensors, in particular the brain activity related to a motor task performed, imagined, or visualized by a subject, using a plurality of magnetoencephalographic or electroencephalographic sensors, when this subject is submitted to a stimulus. The estimation method is based on an MNE criterion in which the coefficients of the covariance matrix of the physiological signals acquired by the different sensors are weighted using the correlation coefficients of these signals with a signal representative of the stimulus.

### TECHNICAL FIELD

The present invention generally relates to the estimation of the electrical activity in a human or animal tissue, and more particularly the localisation of a brain activity from physiological signals, obtained by magnetoencephalography or by electroencephalography. The invention especially applies to the field of functional neural imaging and direct neural control.

### STATE OF PRIOR ART

Functional neural imaging methods are conventionally divided into those based on the metabolic activity such as functional magnetic resonance imaging (fMRI), representing the hemodynamic response, or positron emission tomography (PET), representing modifications in the blood flow, and methods based on the electrophysiological activity, such as electroencephalography (EEG), measuring the electrical brain activity by means of electrodes placed on the subject's scalp, electro-corticography (ECoG) measuring the brain activity by means of electrodes directly placed on the cortical surface, or magnetoencephalography (MEG) measuring the magnetic fields related to the electrical activity in the brain.

The EEG and MEG functional imaging methods have a better time resolution than the fMRI and TEP methods. Furthermore, they are non-invasive unlike electrocorticography. Finally, magnetoencephalography is particularly interesting in that the magnetic signals created by the currents in the brain (mainly ionic currents in dendrites during the synaptic transmission), undergo little or no distortion when they propagate through the cranium.

Besides, electroencephalography and magnetoencephalography are presently the object of considerable research for their potential applications to the direct neural control. Direct neural control or BCI (Brain Computer Interface) enables a direct communication to be established between the brain of a user and an external device (computer, electronic system, effector) with no muscle mediation. Direct neural control uses the association of one or more mental tasks (action imagined by the subject) with one or more controls of the external device. Thus, a hand action imagined by the subject can be associated with a movement of a cursor on a computer screen or the motion of an effector. This technique is very promising especially for people suffering from paralysis.

Whether in the field of functional neural imaging or that of direct control, different methods have been developed to locate a brain activity associated with an (imagined or performed) task from physiological signals acquired by a plurality of sensors. Thus, in the case of EEG, the sensors are electrodes enabling electric potential differences to be acquired at the scalp surface. In the case of MEG, the sensors (SQUIDs) can be magnetometers able to measure the intensity of the magnetic fields and/or planar (or axial) gradiometers able to measure the magnetic field gradient in a given plane. For example, an MEG equipment can combine, in a same location, three simple sensors or even more: a precision magnetometer measuring the intensity and orientation of the magnetic field at a point and two planar gradiometers, perpendicular to each other measuring two components of the magnetic field gradient at this point.

In any case, the object of the abovementioned methods is to locate the brain activity sources from signals acquired by the different sensors. More precisely, if a gridding of the cortex into elementary areas is performed and if x is a vector (of a size M) representative of electric current densities (or source signals) in the different elementary areas and y is a vector (of a size N) representative of the signals acquired by the different sensors, we have the matrix relationship:

y=Ax+b (1)

where A is a matrix of a size N×M, referred to as a lead field matrix which is a function of the considered elementary area and b is a noise sample vector of a size N. The matrix A is usually obtained by simulation from a brain modelling by boundary or finite elements. Thus, the cortex is divided into M elementary areas and each element of the vector x being representative of the electric current density in a voxel, for example taken equal to the norm of the electric current density vector in the voxel in question.

Locating the brain activity amounts to researching the vector x (or, at the very least a vector x insofar as the system (1) is under-determined), from the vector y, that is inverting the relationship (1). For this reason, locating the brain activity from the vector of signals y is sometimes referred to as the “inverse problem” in literature. This inversion is delicate since there is actually an infinity of solutions due to the under-determination of the system (1), the number of sources being considerably higher than the number of sensors. We are then led to make additional hypotheses in order to be able to perform the inversion.

Different solutions to the inverse problem have been suggested in literature, especially the MNE (Minimum Norm Estimate) method, the dSPM (dynamic Statistical Parameter Mapping) method used in locating deep sources, the LORETA (Low Resolution Electromagnetic Tomography) method, beamforming methods especially described in the article by A. Fuchs entitled “Beamforming and its application to brain connectivity” published in the book “Handbook of Brain Connectivity”, V. K. Jirsa, R. A. McIntosh, Springer Verlag, Berlin, pp. 357-378 (2007).

A review of the different abovementioned locating methods can be found in the article by O. Hauk et al. entitled “Comparison of noise-normalized minimum norm estimates of MEG analysis using multiple resolution metrics” published in Neuroimage, Vol. 54, 2011, pp. 1966-1974.

Assuming that the noise is Gaussian and more precisely that the noise samples are independent centred Gaussian random variables which are identically distributed, the solution to (1) can be given by the matrix W which minimizes the square error:

e=∥Wy−x∥² (2)

The equation (2) can also be written as:

e=∥Mx∥²+∥Wb∥²=Tr(MRM^{T})+Tr(WCW^{T}) (3)

where M=WA−I_{M}, I_{M} is the identity matrix of a size M×M, Tr is a trace function, R is the covariance matrix of the source signals, and C is the noise covariance matrix. Minimizing the square error e leads to the solution:

W=RA^{T}(ARA^{T}+C)⁻¹ (4)

and therefore to estimating the location of the brain activity given by:

\hat{x}=RA^{T}(ARA^{T}+C)⁻¹y (5)

The noise covariance matrix can be written as C=σ²I_{N} where σ² is the noise variance and I_{N} is the unit matrix of a size N×N. Similarly, if the different sources are considered as being independent and identically distributed (same strength for all the dipoles), there is R=p·I_{M} where p is the strength of the source signal in all the elementary areas, the relationship (5) can be simplified as:

\hat{x}=A^{T}(AA^{T}+λI_{N})⁻¹y (6)

where λ=σ⁻²/p is an adjustment parameter expressing the significance of the noise to the source signal. The expression (6) is the solution of the abovementioned MNE method.

Whatever the locating method considered, the obtained locating accuracy quickly decreases with the signal-to-noise ratio (λ⁻¹). To overcome this difficulty, the article by Liu et al. entitled “Spatiotemporal imaging of human brain activity using functional MRI constrained magnetoencephalography data: Monte Carlo simulations” published in Proceedings of the National Academy of Sciences of the United States of America, vol. 95, no 15, pp. 8945-8950, July 1998, suggests in particular to use fMRI locating data in order to improve the accuracy of MEG or EEG brain activity localisation. However, this accuracy improvement by data hybridization implies, on the one hand that an fMRI is available for the same task, which is not worth considering for the direct neural control, and, on the other hand, that the electrical/magnetic activity in the brain is correlated with the hemodynamic response. Consequently, it inevitably introduces a bias by orienting the localisation of the MEG or EEG brain activity towards sources which have been detected by fMRI.

The problem underlying the present invention is consequently to provide a method for locating the brain activity associated with a task, from physiological signals, in particular magnetoencephalographic or electroencephalographic signals, which has a higher locating accuracy than that obtained in the state of the art without resorting to a third functional imaging method. More generally speaking, the purpose of the present invention is to provide a better estimation of the electrical activity in the tissue of a human or animal subject from physiological signals acquired when the subject performs or mentions a task, and this without a priori information regarding the location of this electrical activity.

### DISCLOSURE OF THE INVENTION

The present invention is defined by a method for estimating the electrical activity within a tissue of a subject, said electrical activity being associated with a task, performed, imagined, or visualized by the subject when the latter receives a stimulus, wherein acquiring a plurality of physiological signals is performed thanks to a plurality of sensors disposed around the tissue, and wherein:
the correlation coefficients between the different physiological signals and a signal representative of said stimulus are calculated;
the covariance matrix of the physiological signals is calculated over a time window;
the coefficients of the covariance matrix are weighted using the correlation coefficients, so as to penalize, in terms of signal-to-noise ratio, the physiological signals which are weakly correlated with the stimulus, the penalizing diminishing the coefficients related to the physiological signals weakly correlated with the stimulus, when the stimulus is present in the time window and/or increasing these coefficients, when the stimulus is absent from the time window;
the electrical activity is estimated, at least at one point of the tissue, from the physiological signals and the thus-weighted covariance matrix.

The estimation can especially be based on an MNE criterion.

In this case, the covariance matrix is a noise covariance matrix correlated over a time window where the stimulus is absent and the electrical activity in a plurality of elementary areas of the tissue is estimated by means of:

\hat{x}(t)=A^{T}(AA^{T}+p⁻¹\tilde{C}(t))⁻¹y(t)

where \hat{x}(t) is a vector representing the electrical activity in the different elementary areas, y(t) is a vector representing the physiological signals acquired by the sensors, A is a matrix giving the response of the sensors for unit power sources situated in the different elementary areas, p is the real strength of these sources and \tilde{C}(t) is the weighted noise covariance matrix.

Advantageously, the coefficients of the weighted noise covariance matrix are obtained from the noise covariance matrix by means of the following relationship:

$${\tilde{C}}_{ij} ( t ) = {C}_{ij} for i = 1 , … , N , j = 1 , … , N , i \neq j$$

$${\tilde{C}}_{ii} ( t ) = \frac{{C}_{ii}}{1 + {γ}^{2} {χ}_{i}^{2} ( t )} for i = 1 , … , N$$

where the coefficients \tilde{C}ᵢⱼ(t), i=1, . . . , N, j=1, . . . , N are the coefficients of the weighted noise covariance matrix, the coefficients Cᵢⱼ, i=1, . . . , N, j=1, . . . , N are the coefficients of the noise covariance matrix, N is the number of sensors, y is a predetermined real constant, and χᵢ(t), i=1, . . . , N are the correlation coefficients of the physiological signals acquired by the different sensors with the signals representative of the stimulus.

The correlation coefficients χᵢ(t), i=1, . . . , N can be subjected to a normalization prior to the coefficient weighting of the noise covariance matrix.

The estimation method can alternatively use a beamforming for a plurality of elementary areas of the tissue and for a plurality of directions.

In this case, the covariance matrix is calculated over a time window where the stimulus is present and the electrical activity in each elementary area of the tissue is estimated by means of:

\hat{x}_{m,k}(t)=\tilde{D}(t)⁻¹L_{m,k}(L_{m,k}\tilde{D}(t)⁻¹L_{m,k})⁻¹y(t)

where \hat{x}_{m,k}(t) represents the electrical activity in the elementary area situated in a point rₘ and in the direction uₖ, y(t) is a vector representing the physiological signals acquired by the sensors, L_{m,k} is a vector of a size N giving the response of the sensors when a unit power source is at the point rₘ and is oriented in the direction uₖ, and where \tilde{D}(t) is the weighted noise covariance matrix.

Advantageously, the coefficients of the weighted covariance matrix are obtained from the covariance matrix by means of the following relationship:

\tilde{D}ᵢⱼ(t)=Dᵢⱼ|χᵢ(t)∥χⱼ(t)| i=1, . . . ,N, j=1, . . . ,N

where the coefficients \tilde{D}ᵢⱼ(t), i=1, . . . , N, j=1, . . . , N are the coefficients of the weighted covariance matrix, the coefficients Dᵢⱼ, i=1, . . . , N, j=1, . . . , N are the coefficients of the covariance matrix, N is the number of sensors, and χᵢ(t), i=1, . . . , N are the correlation coefficients of the physiological signals acquired by the different sensors with the signal representative of the stimulus.

The correlation coefficients can be obtained by forming a time-frequency or time-scale transform of each physiological signal in order to obtain a plurality of frequency components (Y_{f}(t)) of this signal as a function of time, by calculating the Pearson coefficients (R_{f}(t)) between said frequency components and the signals representative of the stimulus, the correlation coefficient (χ(t)) related to a physiological signal being determined from said obtained Pearson coefficients obtained for this signal.

The correlation coefficient (χ(t)) related to a physiological signal can then be obtained as the extreme value of the Pearson coefficients for the different frequency components of this signal.

### BRIEF DESCRIPTION OF THE DRAWINGS

Further characteristics and advantages of the invention will appear upon reading a preferential embodiment of the invention made with reference to the appended figures among which:

FIG. 1 schematically represents the flow chart of a method for estimating the electrical activity in a tissue according to a first embodiment of the invention;

FIG. 2 schematically represents the flow chart of a method for estimating the electrical activity in a tissue according to a second embodiment of the invention;

FIG. 3 schematically represents an exemplary calculation of a correlation coefficient between a physiological signal and a stimulus signal;

FIG. 4A represents the electrical activity in a human brain, associated with a task, estimated from a method known in the state of the art;

FIG. 4B represents the electrical activity in a human brain, associated with said same task, estimated from an estimation method according to an embodiment of the invention.

### DETAILED DISCLOSURE OF PARTICULAR EMBODIMENTS

A system for acquiring physiological signals from a plurality of sensors disposed around a tissue of interest of a human or animal subject will be thereafter considered, this tissue being the seat of an electrical activity when this subject performs, sees, or visually imagines a task. For the sake of illustration and without a generalization prejudice, we will more particularly consider the case of a magnetoencephalographic acquisition system, being understood that other acquisition systems can be alternatively used, especially an electroencephalographic acquisition system.

The magnetoencephalographic system comprises, in a known manner, a “MEG helmet” placed at a few centimetres from the subject's cranium. This helmet comprises a plurality of sensors situated in different points; each sensor can be made of one or more elementary sensors. The simple sensors can be precision magnetometers and planar (or axial) gradiometers. Alternatively, they can be radial gradiometers such as those described in the article by J. Vrba et al. entitled “Signal processing in magnetoencephalography”, Methods 25, 249-271 (2001). The motions of the subject's head are furthermore recorded and compensated thanks to coils placed in stationary points with respect to the subject's head and generating a magnetic field in a frequency band far from that of the MEG signal.

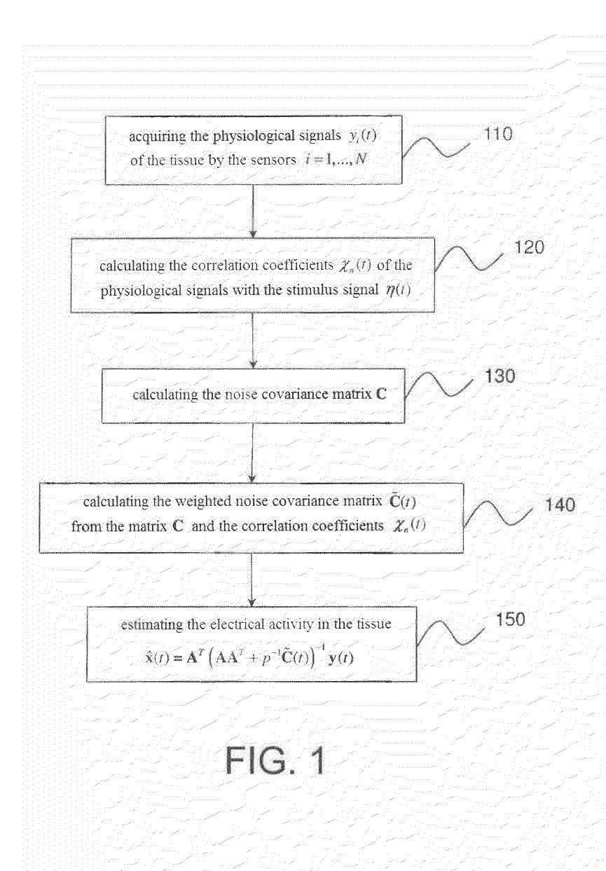

FIG. 1 schematically represents a method for estimating the electrical activity within a subject's tissue, according to a first embodiment of the invention.

In step 110, the subject performs, imagines, or visualizes a task. We could indeed demonstrate that imagining or visualizing a task activated the same brain area as when the subject really performed this same task.

The task in question can be represented by a binary variable η(t) indicating a sensory stimulus, for example a visual or auditory stimulus. For example, when the variable η(t) assumes the value 1, the stimulus is applied and when it assumes the value 0, it is not. When the stimulus is applied, the subject performs, imagines, or visualizes the task in question.

The stimulus can be repeated so as to acquire a plurality of sequences y(t) where y is, as previously defined, the vector (of a dimension N) of the physiological signals acquired by the different sensors at the time t).

In step 120, for each component yₙ(t), n=1, . . . , N, of the vector y(t), a coefficient χₙ representing the correlation between the signal yₙ(t) and the stimulus η(t) is calculated over a given time range, the more significant the correlation between this component and the stimulus in this time range, the higher the coefficient χₙ in terms of absolute value. The correlation coefficient χₙ can be obtained according to different alternatives, as described later. Generally speaking, the coefficient χₙ depends on the time range considered for calculating the correlation and consequently on the time. For this reason, it will be hereinafter noted as χₙ(t).

At the end of the learning phase, a plurality of coefficients χₙ (t), n=1, . . . , N is available, indicating, as a function of time, to which extent the different physiological signals are correlated “with the stimulus”, in other words, are relevant regarding the task in question.

In step 130, the noise covariance matrix C is calculated. This noise covariance matrix is advantageously obtained as the covariance matrix of the physiological signals yₙ(t) when no stimulus is applied and when no task is performed by the subject, in this case when η(t)=0. In other words, the component Cᵢⱼ of the covariance matrix is obtained by:

Cᵢⱼ=E[(yᵢ−E(yᵢ))(yⱼ−E(yⱼ))^{T}] when η(t)=0 (7)

where E(.) means the mathematical expectation. The mathematical expectation E(Z) can be estimated from the average of Z over the time interval during which the stimulus is absent.

In step 140, the coefficients (here the diagonal terms) of the noise covariance matrix are weighted by means of the abovementioned correlation coefficients, so as to penalize, in terms of signal-to-noise ratio, the physiological signals having a weak correlation with this stimulus. This weighting correlatively promotes, in terms of signal-to-noise ratio, the physiological signals having a high correlation with the stimulus. Penalizing results in increased coefficients of the covariance matrix related to the physiological signals weakly correlated with the stimulus, insofar as the covariance matrix is that of the noise covariance matrix.

This weighting is dynamic insofar as the weighting coefficients vary as a function of time. The result of this weighting is a weighted covariance matrix noted as \tilde{C}(t). For example, the coefficients of the matrix \tilde{C}(t) can be obtained from the coefficients of the noise covariance matrix C, in the following way:

$${\tilde{C}}_{ij} ( t ) = {C}_{ij} for i = 1 , … , N , j = 1 , … , N , i \neq j \\ {\tilde{C}}_{ii} ( t ) = \frac{{C}_{ii}}{1 + {γ}^{2} {χ}_{i}^{2} ( t )} for i = 1 , … , N \qquad ( 8 )$$

where γ is a predetermined coefficient. Advantageously, the correlation coefficients χᵢ(t), i=1, . . . , N, are normalized:

$${χ}_{i} ( t ) = \frac{{χ}_{i} ( t ) - min ( {χ}_{i} )}{max ( {χ}_{i} ) - min ( {χ}_{i} )} \qquad ( 9 )$$

so that they take their values in the interval [0,1]. Other weighting functions can be considered by those skilled in the art without departing from the scope of the present invention.

In step 150, an estimation of the electrical activity in the tissue is performed all the time from the vector of the physiological signals y, of the lead field matrix A, as well as the weighting noise covariance matrix, \tilde{C}:

\hat{x}(t)=RA^{T}(ARA^{T}+\tilde{C}(t))⁻¹y(t) (10)

where R is the covariance matrix of the source signals related to the different elementary areas.

It is noted that the lead field matrix A is obtained by simulation, prior to acquiring the physiological signals, by performing a gridding of the tissue into M elementary areas and by calculating by a boundary or finite element method the field at the measuring points of the different sensors. More precisely, from the field generated by a source in an elementary area, the field is calculated in these measuring points. The process is repeated for the M elementary areas so that the M lines of the matrix A are successively obtained.

The covariance matrix R can also be obtained by simulation from the source signals generated in the different elementary areas. Advantageously, theses source signals are assimilated to identically distributed and independent random variables (hypothesis usually confirmed). In this case, the estimation of the electrical activity in the tissue is more simply given by:

\hat{x}(t)=A^{T}(AA^{T}+p⁻¹\tilde{C}(t))⁻¹y(t) (11)

where p is the source signal strength in the elementary area.

The expression (11) implies the inversion of a matrix at each considered time range. In practice, merely performing this inversion every N_{f} time windows (N_{f} being an integer greater than 1) can be enough, by replacing in the expression (8) the correlation coefficients by their respective averages over N_{f} time windows.

In any case, the thus estimated electrical activity can be represented as an image to locate the activity or can be processed, in the case of a brain activity, in order to generate a direct neural control. In the latter case, the processing in question can comprise the integration of the module of the vector \hat{x} on a predetermined area of the brain and the comparison of the integration result with a threshold, or also a spatial correlation of the vector \hat{x} with a predetermined pattern.

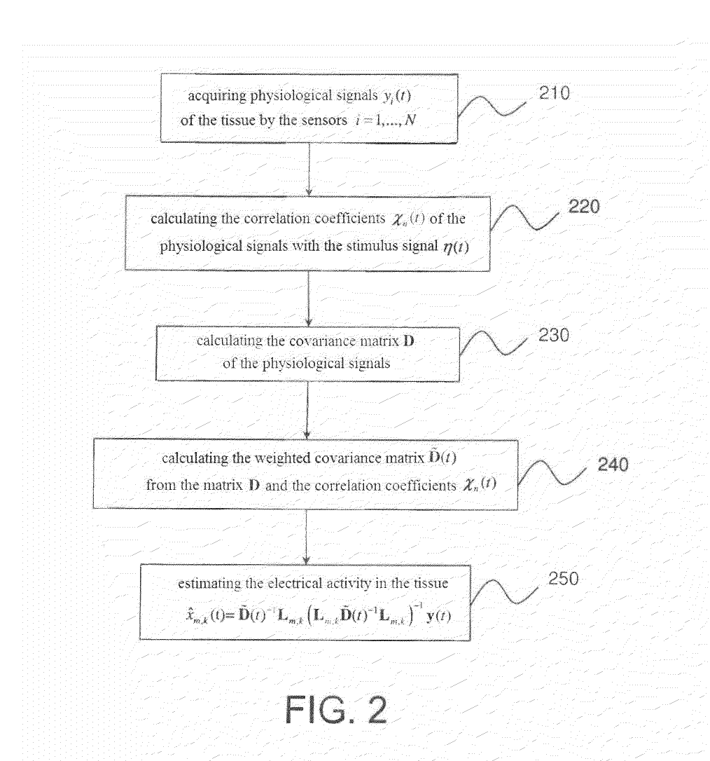

FIG. 2 schematically represents a method for estimating the electrical activity within a subject's tissue, according to a second embodiment of the invention.

Steps 210 and 220 are respectively identical to the previously described steps 110 and 120. In other words, acquiring the physiological signals by means of the different sensors is performed in 210 and calculating the correlation coefficients is performed in 220.

In step 230, the calculation of the covariance matrix of the physiological signals, thereafter noted as D, is carried out. Unlike the first embodiment, calculating the covariance matrix is carried out over a time range in which the stimulus is present:

Dᵢⱼ=E[(yᵢ−E(yᵢ))(yⱼ−E(yⱼ))^{T}] when η(t)=1 (12)

In step 240, the elements of the covariance matrix of the physiological signals are weighted by the correlation coefficients χₙ(t), n=1, . . . , N. More precisely, the components of a matrix \tilde{D}(t) are calculated:

\tilde{D}ᵢⱼ(t)=Dᵢⱼ|χᵢ(t)∥χⱼ(t)| i=1, . . . ,N, j=1, . . . ,N (13)

Thus, in this matrix, the coefficients are weighted as a function of the relevancy of the physiological signals with respect to the task, the signals having little relevance (weak correlation coefficients) being here penalized by reducing the corresponding coefficients in the correlation matrix.

In step 250, forming a plurality of beams is performed, each beam corresponding to an elementary area of the tissue and to a given observation direction.

It can be shown (cf. abovementioned article by A. Fuchs) that the signal coming from an elementary area in a point rₘ (equivalent dipole in the case of MEG) and observed in a direction uₖ is obtained by:

x_{m,k}(t)=w_{m,k}^{T}y(t) (14)

where w_{m,k} is a column vector giving the weights to be assigned to each physiological signal for the beamforming at the reception in the direction uₖ, the vector w_{m,k} being obtained by the expression:

w_{m,k}^{T}=D⁻¹L_{m,k}(L_{m,k}D⁻¹L_{m,k})⁻¹ (15)

in which the vector L_{m,k} of a size N is the vector of the field measured by the N sensors when a source is at the point rₘ and is oriented in the direction uₖ. This vector is obtained by simulation from a propagation model in the tissue, in a manner known per se.

In the present embodiment, the contributions of the physiological signals are weighted by the correlation coefficients, more precisely the electrical activity of the tissue at the point rₘ in the direction uₖ is estimated by:

\hat{x}_{m,k}(t)=\tilde{w}_{m,k}^{T}(t)y(t) (16)

where \tilde{w}_{m,k}^{T}(t)=\tilde{D}(t)⁻¹L_{m,k}(L_{m,k}\tilde{D}(t)⁻¹L_{m,k})⁻¹ (17)

The expression (17) requires the inversion of the weighted covariance matrix \tilde{D}(t). As in the first embodiment, this inversion can only be performed every N_{f} time windows, the correlation coefficients in the expression (13) being then replaced by their respective averages over these N_{f} windows.

The inversion of the matrix \tilde{D}(t) can be performed after diagonalization. The eigenstates for which the eigenvalues are significant (higher than a threshold value) are those which are relevant for the task performed or imagined by the subject. The conditioning the matrix \tilde{D}(t) can be improved using an adjustment parameter when the eigenvalues are lower than a determined threshold.

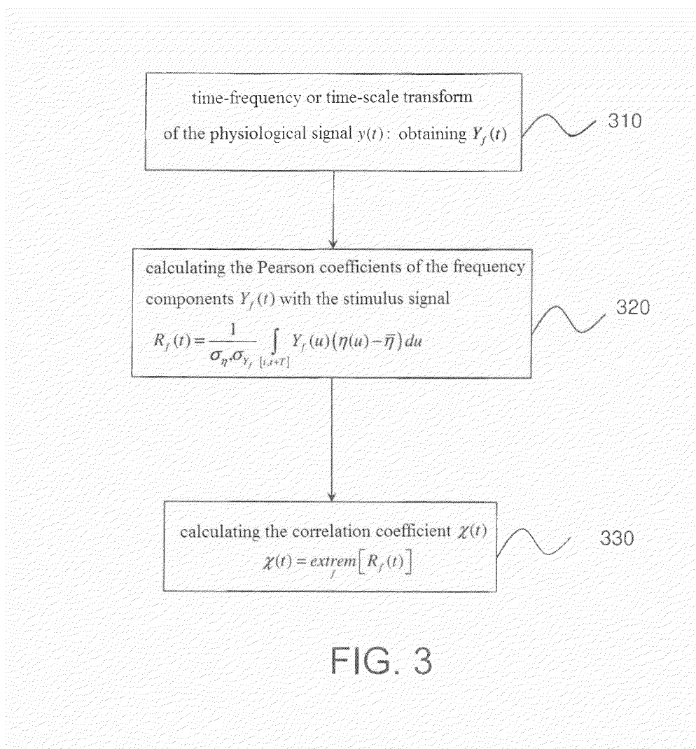

FIG. 3 schematically represents an exemplary calculation of the correlation coefficient of a physiological signal with a stimulus signal, that is, more precisely, of a signal yₙ(t) provided by a sensor and the stimulus η(t), such as above-defined. The physiological signal yₙ(t) will be simply noted afterwards as y(t), the calculation being identical whatever the sensor.

In a first step 310, a time-frequency transform or a time-scale transform of the physiological signal y(t) is calculated. The time-frequency transform can be for example a weighting short-term Fourier transform using a sliding time window, the time-scale transform can be a continuous wavelet transform (CWT) in a manner know per se. The Morlet-Gabor wavelet or a so-called Mexican hat wavelet can be used to this end.

In any case, a frequency representation is obtained as a function of time, Y_{f} (t), of the physiological signal y(t), the term Y_{f}(t) giving the “instant” frequency component (or more precisely in a frequency band) of the signal y(t).

If need be, these frequency components can be smoothed over time by a low-pass filtering, for example by means of a moving average with a forgetting coefficient.

In a second step 320, the Pearson coefficient R_{f} of each frequency component Y_{f} is calculated in the following way:

$${R}_{f} ( t ) = \frac{1}{{σ}_{η} \cdot {σ}_{{Y}_{f}}} {\int }_{[ t , t + T ]} {Y}_{f} ( u ) ( η ( u ) - \overline{η} ) d u \qquad ( 18 )$$

the integration over the sliding window [t, t+T] can of course be performed by means of a discrete summation and the calculation being performed for a discrete set of the frequency. σ_{η} and σ_{Y}_{_{f}} respectively represent variance of the stimulus η and of the frequency component Y_{f} and \tilde{η} is the average value of η on the sliding window in question.

In a third step 330, the correlation coefficient χ(t) of the physiological signal y(t) with the stimulus η(t) is calculated from the Pearson coefficients R_{f}(t). For example, for χ(t), the extreme value of R_{f}(t) in the frequency range of interest can be taken:

$$χ ( t ) = {extrem}_{f} [ {R}_{f} ( t ) ] \qquad ( 19 )$$

It is noted that the extreme value of a function is that of the maximum value and of the minimum value which is the largest in absolute value. Similarly, since it intervenes previously only in absolute value, the correlation coefficient can be chosen as the maximum of |R_{f}(t)| in the frequency range of interest. Other calculation alternatives of the correlation coefficient can be considered by those skilled in the art, for example the integration of |R_{f}(t)| or of (R_{f}(t)) on the frequency range of interest.

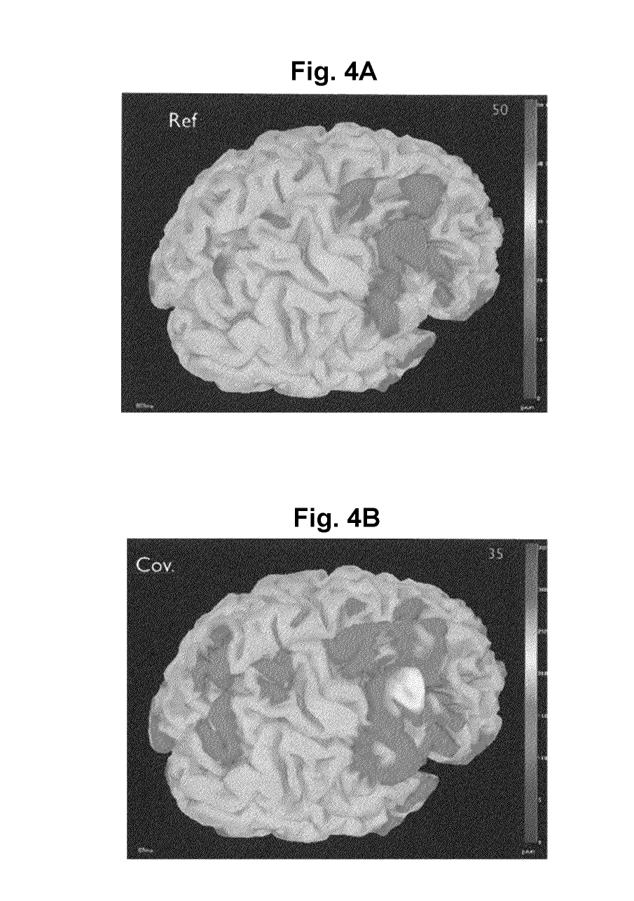

FIG. 4A represents an image of the electrical activity in a human subject's brain, corresponding to a task performed by this subject, such as obtained by the MNE estimation method, more precisely as estimated by means of the expression (6).

FIG. 4B represents the electrical activity in the brain in question, again for the same task performed, but obtained by means of the estimation method according to the first embodiment of the invention, more precisely as estimated by means of the expression (11).

It is noticed that the brain activity represented in FIG. 4B is better focused than that represented in FIG. 4A. Thus, it was possible to refine the localisation of the brain activity without providing locating data coming from another functional imaging technique (fMRI for example), but simply by adding to the MNE reference technique an a priori information (correlation measurement) in the sensor space. These conclusions remain valid for other tasks such as the imagination of a leg motion, implying other motor cortical areas.

1. A method for estimating the electrical activity within a tissue of a subject, said electrical activity being associated with a task, performed, imagined, or visualized by the subject when the latter receives a stimulus, wherein acquiring a plurality of physiological signals is performed thanks to a plurality of sensors disposed around the tissue, said method being characterised in that:
the correlation coefficients between the different physiological signals and a signal representative of said stimulus are calculated;
the covariance matrix of the physiological signals is calculated over a time window;
the coefficients of the covariance matrix are weighted using the correlation coefficients, so as to penalize, in terms of signal-to-noise ratio, the physiological signals which are weakly correlated with the stimulus, the penalizing diminishing the coefficients related to the physiological signals weakly correlated with the stimulus, when the stimulus is present in the time window and/or increasing these coefficients, when the stimulus is absent from the time window;
the electrical activity is estimated, at least at one point of the tissue, from the physiological signals and the thus-weighted covariance matrix.

2. The method for estimating the electrical activity within a tissue according to claim 1, characterised in that the estimation is based on an MNE criterion.

3. The method for estimating the electrical activity within a tissue according to claim 2, characterised in that the covariance matrix is a noise covariance matrix calculated over a time window where the stimulus is absent and in that the electrical activity in a plurality of elementary areas of the tissue is estimated by means of:
\hat{x}(t)=A^{T}(AA^{T}+p⁻¹\tilde{C}(t))⁻¹y(t)
where \hat{x}(t) is a vector representing the electrical activity in the different elementary areas, y(t) is a vector representing the physiological signals acquired by the sensors, A is a matrix giving the answer of the sensors for unit power sources situated in the different elementary areas, p is the real strength of these sources and \tilde{C}(t) is the weighted noise covariance matrix.

4. The method for estimating the electrical activity within a tissue according to claim 3, characterised in that the coefficients of the weighted noise covariance matrix are obtained from the noise covariance matrix by means of the following relationship:
$${\tilde{C}}_{ij} ( t ) = {C}_{ij} for i = 1 , … , N , j = 1 , … , N , i \neq j$$
$${\tilde{C}}_{ii} ( t ) = \frac{{C}_{ii}}{1 + {γ}^{2} {χ}_{i}^{2} ( t )} for i = 1 , … , N$$
where the coefficients \tilde{C}ᵢⱼ(t), i=1, . . . , N, j=1, . . . , N are the coefficients of the weighted noise covariance matrix, the coefficients Cᵢⱼ, i=1, . . . , N, j=1, . . . , N are the coefficients of the noise covariance matrix, N is the number of sensors, γ is a predetermined real constant and χᵢ(t), i=1, . . . , N are the correlation coefficients of the physiological signals acquired by the different sensors with the signal representative of the stimulus.

5. The method for estimating the electrical activity within a tissue according to claim 4, characterised in that the correlation coefficients χ(t), i=1, . . . , N are subjected to a normalization prior to the coefficient weighting of the noise covariance matrix.

6. The method for estimating the electrical activity within a tissue according to claim 1, characterised in that the estimation method uses a beamforming for a plurality of elementary areas of the tissue and for a plurality of directions.

7. The method for estimating the electrical activity within a tissue according to claim 6, characterised in that the covariance matrix is calculated over a time window where the stimulus is present and in that the electrical activity in each elementary area of the tissue is estimated by means of:
\hat{x}_{m,k}(t)=\tilde{D}(t)⁻¹L_{m,k}(L_{m,k}\tilde{D}(t)⁻¹L_{m,k})⁻¹y(t)
where \hat{x}_{m,k}(t) represents the electrical activity in the elementary area situated in a point rₘ and in the direction uₖ, y(t) is a vector representing the physiological signals acquired by the sensors, L_{m,k} is a vector of a size N giving the answer of the sensors when a unit power source is at the point rₘ and is oriented in the direction uₖ, and where \tilde{D}(t) is the weighted noise covariance matrix.

8. The method for estimating the electrical activity within a tissue according to claim 7, characterised in that the coefficients of the weighted covariance matrix are obtained from the covariance matrix by means of the following relationship:
\tilde{D}ᵢⱼ(t)=Dᵢⱼ|χᵢ(t)∥χⱼ(t)| i=1, . . . ,N, j=1, . . . ,N
where the coefficients \tilde{D}ᵢⱼ(t), i=1, . . . , N, j=1, . . . , N are the coefficients of the weighted covariance matrix, the coefficients Dᵢⱼ, i=1, . . . , N, j=1, . . . , N are the coefficients of the covariance matrix, N is the number of sensors and χᵢ(t), i=1, . . . , N are the correlation coefficients of the physiological signals acquired by the different sensors with the signal representative of the stimulus.

9. The method for estimating the electrical activity within a tissue according to claim 1, characterised in that the correlation coefficients are obtained by performing a time-frequency or time-scale transform of each physiological signal in order to obtain a plurality of frequency components (Y_{f}(t)) of this signal as a function of time, by calculating the Pearson coefficients (R_{f}(t)) between said frequency components and the signal representative of the stimulus, the correlation coefficient (χ(t)) related to a physiological signal being determined from said obtained Pearson coefficients for this signal.

10. The method for estimating the electrical activity within a tissue according to claim 9, characterised in that the correlation coefficient (χ(t)) related to a physiological signal is obtained as the extreme value of the Pearson coefficients for the different frequency components of this signal.
